(19) United States
(12) Patent Application Publication
(10) Pub. No.: US 2003/0044428 A1
(43) Pub. Date: Mar. 6, 2003

(54) Method for treating an HIV-infected individual by combining immunization with structured interruption of anti-retroviral treatment

(76) Inventors: Ronald B. Moss, San Diego, CA (US); Dennis J. Carlo, Rancho Santa Fe, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/056,420

(22) Filed: Jan. 24, 2002

Related U.S. Application Data
(60) Provisional application No. 60/264,476, filed on Jan. 26, 2001.

(51) Int. Cl. A61K 39/21; A61K 31/00

(52) U.S. Cl. 424/208.1; 514/1

(57) ABSTRACT

The invention provides a method for the treatment of HIV infected individuals. The method is practiced by combining immunization with an HIV immunogenic composition with structured cycles of anti-retroviral treatment and withdrawal from treatment.

This application claims the benefit of U.S. Provisional Application No. 60/264,476, filed Jan. 26, 2001, which is incorporated herein by reference in its entirety.

### BACKGROUND OF THE INVENTION

1. Field of the Invention

The invention relates generally to the fields of medicine and immunology and, more specifically, to methods of treating HIV-infected individuals by combining immunization with an HIV immunogenic composition with structured cycles of anti-retroviral treatment and withdrawal from treatment.

2. Background Information

The introduction of potent anti-retroviral drug therapy has significantly improved the ability of many HIV infected individuals to maintain suppression of HIV replication to low levels for an extended period of time. These effects have translated into a dramatic reduction in AIDS-related opportunistic infections and death in those with access to the medications.

Unfortunately, most effective anti-retroviral drug regimens require daily treatments with multiple drugs, which are both complex and expensive. Additionally, anti-retroviral drug regimens are associated with significant toxicities with long term use, including increases in serum cholesterol and triglycerides, cardiotoxicity and insulin resistance. These factors have led to difficulties with treatment compliance. Furthermore, prolonged anti-retroviral drug treatment often results in outgrowth of drug resistant variants.

It is well established that once anti-retroviral treatment is stopped, patients usually rebound with viral loads at least as high and often higher than original levels. One reason for the inability of infected patients, and particularly chronically infected patients, to control viral replication after drug withdrawal could be that they no longer have sufficient levels of HIV-specific immune cells to respond to the autologous virus. More particularly, the number of HIV-specific CD4 T helper cells is inadequate for effective conversion of CD8 T cells into potent killer cells.

Structured Treatment Interruption (STI), which involves supervised cycles of intermittent withdrawal and reinitiation of anti-retroviral drug therapy, has recently been proposed as a method of overcoming some of the disadvantages of long-term daily anti-retroviral therapy for the treatment of HIV-infected individuals. STI has also been predicted to provide the additional benefit of allowing autologous virus levels to increase during the drug withdrawal period, leading to a stimulation of the immune system that provides control of viral load. However, STI has not consistently proven useful in controlling viral load during withdrawal from anti-retroviral therapy, especially in chronically infected individuals.

Thus, there exists a need for improved therapeutic methods for treating HIV-infected individuals, and particularly for treating chronically infected individuals, which provide the benefits of intermittent withdrawal from anti-retroviral therapy, while controlling viral load at acceptably low levels during withdrawal from anti-retroviral therapy. The present invention satisfies this need, and provides related advantages as well.

### SUMMARY OF THE INVENTION

The invention provides a method of treating an HIV-infected individual. The method is practiced by

(a) treating an HIV-infected individual with at least one anti-retroviral compound;

(b) immunizing said individual with an HIV immunogenic composition;

(c) withdrawing treatment with said anti-retroviral compound;

(d) reinitiating treatment with at least one anti-retroviral compound;

(e) repeating step (c) at least once; and

(f) optionally repeating step (d) at least once.

### DETAILED DESCRIPTION OF THE INVENTION

The invention provides an improved method for the treatment of HIV-infected individuals. By incorporating immunization with an HIV immunogenic composition into structured cycles of anti-retroviral treatment and withdrawal from treatment, the invention method is advantageous in maintaining a low viral load in the HIV-infected individual during withdrawal of anti-retroviral treatment, and in reducing the toxicity, cost and inconvenience of continuous anti-retroviral treatment.

The invention method is practiced by immunizing an HIV-infected individual with an HIV immunogenic composition and treating the individual with at least one effective anti-retroviral compound. When viral load is sufficiently lowered, treatment with the anti-retroviral compound is withdrawn. After a suitable time period, which can be a predetermined time period or after viral load rebounds to a predetermined level, anti-retroviral treatment is reinitiated. When viral load is again sufficiently lowered, anti-retroviral treatment is withdrawn and, if deemed appropriate, reinitiated. Cycles of anti-retroviral treatment and withdrawal can optionally be repeated one or more additional times, and immunizations can optionally be repeated one or more additional times, such that viral load is maintained at an acceptably low level for a suitable period of time in the absence of continuous anti-retroviral treatment. It is contemplated that for those individuals whose CD4 levels are sufficiently high during withdrawal of anti-retroviral therapy, anti-retroviral therapy need not be reinitiated to maintain acceptably low viral load.

An important component of the mechanism underlying the effectiveness of the invention method is believed to be the effective stimulation of both CD4 and CD8 anti-HIV immune responses by immunization with an HIV immunogenic composition. Patients undergoing continuous anti-retroviral treatment, although often effectively maintaining low viral loads, generally have reduced CD4 and CD8 T cell responses to the virus. During a first period of structured withdrawal from anti-retroviral treatment, HIV load begins to rebound. In an immune competent patient, this autologous virus should induce a CD8 killer cell response capable of destroying the newly formed virus. However, as a result of virally induced reduction of effective CD4 T helper cell activity, the cytotoxic activity of these CD8 killer cells is not sufficiently strong or prolonged to keep viral load at an acceptably low level without reinitiating anti-retroviral treatment.

As disclosed herein, immunization with a suitable HIV immunogenic composition induces specific and potent anti-HIV CD4 T helper cell activity, which can then enhance CD8 killer cells. Thus, during withdrawal from anti-retroviral treatment, the activity of these CD8 killer cells, as enhanced by vaccine stimulated CD4 T helper cells, serves to maintain HIV viral load at an acceptably low level.

Based on the disclosure herein, the skilled person can determine an appropriate HIV immunogenic composition to stimulate an effective HIV-specific CD4 response, as well as determine appropriate lengths and numbers of treatment withdrawal periods to stimulate an effective CD8 response against autologous HIV that controls HIV viral load.

Individuals contemplated for treatment by the methods of the invention method include both acutely HIV-infected individuals (i.e. individuals infected for less than about 12 months, such as less than about 6 months) and chronically HIV-infected individuals (i.e. individuals infected for more than about 12 months).

It is generally observed that viral load is several logs higher in chronically infected individuals than in acutely infected individuals. It follows that reduction of viral load in chronically infected patients will, in general, require more cycles of structured anti-retroviral therapy and withdrawal than for acutely infected individuals.

As described in the Example, the baseline post-immunization CD4 T helper cell response appears to be correlated with the decrease in viral load peaks between the first and second periods of withdrawal of anti-retroviral therapy. Accordingly, the skilled person can evaluate the CD4 T helper cell responses in individual patients following immunization as a means of determining which individuals are likely to benefit most from treatment by the invention method.

HIV-infected individuals amenable to treatment by the invention method can be either symptomatic or asymptomatic at the time anti-retroviral treatment or immunization is initiated. The method is contemplated for treatment of both adults and children of either gender, including pregnant women.

The steps of initially treating the individual with at least one anti-retroviral compound and of immunizing the individual with an HIV immunogenic composition can take place simultaneously or sequentially, in either order, and for any duration. For example, anti-retroviral treatment can be initiated several years, months or weeks prior to the first immunization. Alternatively, the first immunization can be initiated prior to anti-retroviral treatment. Booster immunizations, if desired, can take place during initial anti-retroviral treatment, during a structured treatment interruption, or during subsequent anti-retroviral treatment. The skilled person can determine an appropriate temporal order and duration for initial treatment with an anti-retroviral compound and for immunizing the individual.

Suitable anti-retroviral compounds and treatment regimens for use in the methods of the invention are those that are able to reduce HIV viral load to a low level and to maintain HIV viral load at the low level for an extended period. Particularly suitable compounds and regimens are those that are able to reduce plasma viral load to less than about 5000 copies/ml, including less than about 2500 or 1000 copies/ml, such as less than 750, 500, 250, 100 or 50 copies/ml prior to the first treatment withdrawal.

The same anti-retroviral compounds and regimens as used initially, or different compounds and regimens, can be used to restore viral load to similarly low levels, or lower levels, when anti-retroviral treatment is reinitiated after a treatment withdrawal. Anti-retroviral compounds and regimens for reducing HIV viral load, and for maintaining such reduced viral load for a period of several days, weeks, months or longer, are well known in the art.

Contemplated anti-retroviral compounds can act by any mechanism that affects the HIV replicative cycle. Such compounds include, for example, compounds that inhibit protease activity, reverse transcriptase activity, ribonucleotide reductase activity, viral adsorption, viral entry, virus-cell fusion, viral assembly and disassembly, proviral DNA integration, viral mRNA transcription, and other processes, as well as combinations of compounds with the same or different mechanisms of action. Effective compounds and combinations and treatment parameters are well known in the art and described, for example, in WO 00/45844 and in De Clerq, Curr. Med. Chem. 8:1543-1572 (2001).

Exemplary protease inhibitors include indinavir sulfate (Crixivan™), saquinavir (Invirase® and Fortovase®), ritonavir (Norvir®), ABT-378, Nelfinavir (Viracept®), GW141, Tipranavir, PD 178390, BMS-23632, DMP-450 and JE 2147. Other contemplated protease inhibitors include derivatives of hydroxyethylamine, hydroxyethylene, hydroxyethylurea and norstantine.

Reverse transcriptase inhibitors include, for example, nucleoside analogs, such as AZT (zidovudine (Retrovir™)), ddC (zalcitabine (Hivid®)), 3TC (lamivudine (Epivir™)), F-ddA (lodenosine), D4T (stavudine (Zerit®)), and other 2′,3′-dideoxynucleoside analogs. Other nucleoside reverse transcriptase inhibitors include adefovir (Preveon®), abacavir (1592U89) and lubocavir. Non-nucleoside reverse transcriptase inhibitors (NNRTIs) include nevirapine (Viramune™), delaviridne (Rescriptor®), efavirenz (Sustiva®), and second-generation NNRTIs such as capravirine and quinoxaline, quinazolinone, PETT and emivrine analogs.

Exemplary ribonucleotide reductase inhibitors include hydroxyurea, guanazole, dihydroxybenzoyl derivatives, thiosemicarbazone derivatives, A1110U, MdCDP, dFdCDP, Cl—F-ara-A, DDC and A723U.

Viral adsorption inhibitors generally bind to the viral envelope glycoprotein gp120, and include, for example, polysulfates, polysulfonates, polyoxometalates, zintevir, negatively charged albumins and cosalane analogs.

HIV entry inhibitors generally act by blocking the viral co-receptors CXCR4 or CCR5, and include, for example, bicylams (AMD3100), polyphemusins (T22), TAK-779 and MIP-1α LD78 β-isoform.

Integrase inhibitors affect proviral DNA integration and include, for example, AR177, Zintenvir®, L-chicoric acid, and diketo acids (L-731,988).

Virus-cell fusion inhibitors generally bind to the viral glycoprotein gp41. Fusion inhibitors include, for example, pentafuside, siamycins, betulinic acid derivatives, T-20 (DP-178) and T-1249 (DP-107).

Viral assembly and disassembly inhibitors include, for example, NCp7 zinc finger-targeted agents such as 2,2′-dithiobisbenzamides (DIBAs), azacarbonamine (ADA) and NCp7 peptide mimetics.

Compounds that inhibit the HIV mRNA transcription/transactivation process include, for example, fluoroquinolone K-12, Streptomyces product EM2487, temacrazine and CGP64222.

Other exemplary anti-retroviral compounds include cytokine and chemokines inhibitors; antisense oligonucleotides (e.g. GPI-2A; ISIS-13312; GEM-132; and GEM-92); RNA-cleaving DNA enzymes (DNAzymes) (e.g. DzV3-9); ribozymes and decoy RNA.

The particular anti-retroviral compounds and combinations used can be determined by the clinician and varied during the treatment protocol, as needed, depending on the response of the individual and the observed side effects. It will be appreciated that if new drugs are subsequently developed with improved safety or efficacy, or which are less expensive, these can be used during cycles of anti-retroviral therapy.

Effective dosages of anti-retroviral compounds are well known in the art, or can be readily determined by the skilled person. The particular treatment regimen will depend, for example, on the nature, toxicity and bioactivity of the compound; on concurrently administered therapies; on the weight, age, gender and health of the individual; on the immune status of the individual; and on the ability of the individual to comply with the regimen. Administration can be by any route suitable for the particular compound or combination, with oral administration preferred.

In the methods of the invention, the HIV-infected individual is immunized with an HIV immunogenic composition. A suitable HIV immunogenic composition induces an HIV antigen-specific CD4+ T helper cell response. HIV antigen-specific CD4+ T helper cell responses can be evidenced by the induction of a lymphocyte proliferative response (LPR) to one or more conserved HIV antigens (such as p24) and/or induction of strong anti-HIV humoral (antibody) responses, as described in the Example and in PCT publication WO 00/67787. As shown in Table 1, induction of a LPR in response to immunization can be evidenced, for example, by a p24 lymphocyte stimulation index (LSI) following immunization of several-fold higher than the pre-immunization LSI.

A suitable HIV immunogenic composition can also induce HIV antigen-specific production of the β-chemokines MIP-1α, MIP-1β and RANTES. Methods of determining the induction of β-chemokine production are known in the art (see, for example, PCT publication WO 00/67787).

An HIV immunogenic composition includes an HIV immunogen, optionally includes an adjuvant, and optionally further includes an immunostimulatory molecule. A suitable HIV immunogen can be a whole-killed HIV virus, (i.e. an intact, inactivated HIV virus), or include or encode any subunit or subunits thereof (e.g. products encoded by the gag genes (p55, p39, p24, p17 and p15), the pol genes (p66/p51 and p31-34), or the transmembrane glycoprotein gp41). The HIV immunogen can be administered in any form, such as as a viral particle, as a protein or as an encoding nucleic acid molecule.

A contemplated HIV immunogen suitable for use in the methods of the invention is a whole-killed HIV virus, which can be intact or devoid of outer envelope protein gp120. Viral killing can be performed by methods known in the art, including treatment with beta-propiolactone and/or gamma irradiation. Whole-killed HIV contains the more genetically conserved parts of the virus (e.g. p24 and gp41) in order to induce cell-mediated responses to a wide range of heterologous viruses. Methods for preparing whole-killed HIV particles are described, for example, in Richieri et al., Vaccine 16:119-129 (1998), and U.S. Pat. Nos. 5,661,023 and 5,256,767.

An exemplary whole-killed HIV immunogen is derived from virus with a clade A envelope and clade G gag, more particularly the HZ321 HIV-1 isolate from an individual infected in Zaire in 1976, which is described in Choi et al., AIDS Res. Hum. Retroviruses 13:357-361 (1997).

Methods of removing the outer envelope proteins of isolated HIV particles are also known in the art. One such method is repeated freezing and thawing of the virus in conjunction with physical methods that cause the swelling and contraction of the viral particles. Other physical or non-physical methods, such as sonication, can also be employed alone or in combination.

Another suitable HIV immunogen is an inactivated protease-defective viral HIV-1 particle, such as described in U.S. Pat. No. 6,328,976. An inactivated protease-defective viral HIV-1 particle can optionally have one or more mutations in the genes encoding Env gp120 or gp41, the Pol protease, Nef, or Vpr.

Other suitable HIV immunogens and their use are known in the art and reviewed, for example, in Peters, Vaccine 20:688-705 (2002). Exemplary HIV immunogens can contain a recombinant envelope protein (e.g. VaxSyn™) or envelope peptide (e.g. PCLUS 3-18MN and PCLUS 6.1-18MN); one or more HIV-1 genes (e.g. gag, pol, env, nef) incorporated into recombinant canarypox virus (e.g. vCP1452, ALVAC1452, ALVAC-HIV, vCP205), vaccinia virus (e.g. NYVAC), coxackie virus or vesicular stomatitis virus; or Tat protein or Tat toxoid.

DNA-based HIV immunogens and their use are also known in the art and reviewed, for example, in Peters, Vaccine 20:688-705 (2002). Such immunogens encode one or several HIV genes, and can optionally encode the entire HIV genome. If the immunogen encodes an entire HIV genome, at least one gene will generally encode a defective gene product to ensure that only non-infectious particles are produced.

The skilled person can determine the amount of immunogen to use for a particular individual, based on factors that include body weight, the nature of the HIV immunogen, and the presence and nature of other components in the composition. For example, an immunogenic composition formulated for a single administration can contain between about 1 to 1000 μg of HIV immunogen, such as between about 2 to 500 μg of HIV immunogen, including about 5 to 100 μg, or about 10 to 50 μg of HIV immunogen.

An HIV immunogenic composition can include the immunogen formulated in a physiologically acceptable buffer, such as saline. Optionally, the composition can further contain an adjuvant. An adjuvant is a substance which, when added to an immunogenic agent, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as polysytrene, starch, polyphosphazene and polylactide/polyglycosides. Adjuvants can also include, for example, squalene mixtures (SAF-I), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, oligolysine, lipopeptides and immunostimulating complexes (ISCOMs), and the like. Suitable adjuvants for administration to humans and other mammals are well known in the art and are reviewed, for example, by Warren and Chedid, CRC Critical Reviews in Immunology 8:83 (1988).

An exemplary HIV immunogenic composition for use in the methods of the invention is REMUNE™, which is a combination of whole-killed HIV virus devoid of outer envelope protein gp120 and Incomplete Freund's Adjuvant (IFA) (see, for example, Levine et al., J. Acquir. Immune Defic. Syndr. Hum. Retrovirol. 11:351-364 (1996); Limsuwan et al., Vaccine 16:142-149 (1998); Churdboonchart et al., Clin. Diagn. Lab. Immunol. 7:728-733 (2000)).

An HIV immunogenic composition can further contain one or more immunostimulatory molecules that augment the effects of the immunogen. For example, the composition can contain an immunostimulatory sequence, or ISS. An ISS is a nucleic acid molecule having a nucleotide sequence that contains at least one unmethylated CpG motif that is capable of enhancing the immune response in a mammal when administered in combination with an antigen. Immunostimulatory sequences are described, for example, in PCT publication WO 98/55495, and their uses in HIV immunogenic compositions are described in PCT publication WO 00/67787.

An ISS can contain, for example, at least one sequence consisting of 5′-Cytosine, Guanine, Pyrimidine, Pyrimidine-3′, such as the hexameric motif 5′-Purine, Purine, Cytosine, Guanine, Pyrimidine, Pyrimidine-3′, such as the motif 5′-GACGTT-3′ (SEQ ID NO: 1). An ISS can also contain, for example, either the octameric motif 5′-Purine, Purine, Cytosine, Guanine, Pyrimidine, Pyrimidine, Cytosine, Cytosine-3′ or 5′-Purine, Purine, Cytosine, Guanine, Pyrimidine, Pyrimidine, Cytosine, Guanine-3′, such as the sequence 5′-AACGTTCG-3′ (SEQ ID NO: 2). Exemplary ISS sequences that enhance HIV-specific Th1 cytokine (IFN-Υ) and humoral responses (IgG2 antibodies), and also enhance both non-specific and HIV-specific β-chemokine production, include the oligonucleotide sequences 5′ TCCATGACGTTCCTGACGTT 3′ (SEQ ID NO: 3); 5′ TGACTGTGAACGTTCGAGATGA 3′ (SEQ ID NO: 4); and 5′-TCGTCGCTGTTGTCGTTTCTT-3′ (SEQ ID NO: 5), as described in PCT publication WO 00/67787.

An ISS can be, for example, a synthetic oligonucleotide, a naturally occurring nucleic acid molecule of any species, or a vector, and can be either DNA or RNA. An ISS can contain either natural or modified nucleotides or natural or unnatural nucleotide linkages. Modifications known in the art, include, for example, modifications of the 3′OH or 5′OH group, modifications of the nucleotide base, modifications of the sugar component, and modifications of the phosphate group. An unnatural nucleotide linkage can be, for example, a phosphorothioate linkage in place of a phosphodiester linkage, which increases the resistance of the nucleic acid molecule to nuclease degradation. Various modifications and linkages are described, for example, in PCT publication WO 98/55495.

The amount of ISS to use in an immunogenic composition can be determined by the skilled person. Generally, the amount of a nucleic acid molecule containing an ISS included in an immunogenic composition will be from about 0.1 μg/ml to about 1 mg/ml, such as from about 1 μg/ml to about 500 μg/ml, including about 5 μg/ml, 25 μg/ml, 50 μg/ml, 100 μg/ml or about 250 μg/ml.

The skilled person understands that other immunostimulatory components can optionally be included in an HIV immunogenic composition, or optionally administered together with administration of an HIV immunogenic composition. Such components are known in the art and include, for example, cytokines, such as IL-12, IL-2 and GM-CSF, and heat shock proteins, such as HSP70.

An individual treated by the invention method can optionally be administered two or more different HIV immunogenic compositions, either simultaneously or sequentially. For example, an individual can be administered an immunogenic composition that contains a viral particle immunogen and a first adjuvant, and another that contains a nucleic acid or peptidic immunogen and a second adjuvant. Likewise, a single immunogenic composition can contain more than one type of HIV immunogen, such as any combination of a viral particle, a nucleic acid and a peptidic immunogen, formulated with a single type of adjuvant. The skilled person can determine an appropriate immunogenic composition or combination of immunogenic compositions for use in the treatment method.

The duration of the first treatment withdrawal can be determined based on the period of time during which viral load is maintained at an acceptably low level. Alternatively, the duration of the first treatment withdrawal can be a predetermined period. The withdrawal period will generally be at least 1 week, such as at least about 2, 4, 6, or 8 weeks, and can be about 10, 12, 16, 20, 30, 40 weeks or longer. As shown in the Example, an exemplary anti-retroviral treatment withdrawal period is 8 weeks. In patients with higher levels of CD4 T lymphocytes, such as CD4 counts of at least about 200 cells/mm³ or at least about 300 cells/mm³, it is anticipated that the duration of treatment withdrawal can be extended for long periods, or indefinitely, while maintaining suitably low viral load.

Low viral load is correlated with the effectiveness of CD8 stimulation during treatment withdrawal. CD8 stimulation can be determined by methods known in the art. Exemplary methods include direct cytolytic assays, as well as ELISA and ELISPOT assays for CD8-specific IFN-Υ production, which is correlated with CD8 cytolytic activity (see, for example, WO 00/67787).

Generally, anti-retroviral drug treatment need not be reinitiated until viral load has rebounded to a predetermined level. The predetermined level at which anti-retroviral treatment is reinitiated can be determined by the skilled person, but will generally be at a viral load of greater than about 20,000 copies/ml, such as greater than about 50,000 or greater than about 100,000 copies/ml.

Second, third, fourth, or subsequent treatment withdrawal periods can be of the same duration, shorter or longer than the first withdrawal period. It is contemplated that the invention method may be effective in allowing second or subsequent treatment withdrawal periods to be extended for longer periods of time, and perhaps indefinitely, while maintaining viral load at an acceptably low level.

The invention method is preferably practiced with at least 2 cycles of treatment withdrawal, although in some individuals additional benefits in controlling viral load can be observed with 3, 4, 5 or more cycles. In view of the advantages in lowering treatment cost and toxicity by withdrawal from anti-retroviral treatment, it is beneficial to practice the invention with the minimal number of cycles needed to maintain viral load at an acceptably low level without continued anti-retroviral treatment. However, there is no contemplated upper limit for the number of treatment and withdrawal cycles that can be used to treat an individual.

In comparison with HIV treatment methods used in the art, such as structured anti-retroviral treatment without immunization, the invention method provides several advantages. For example, by practice of the invention method, viral load can be reduced to a lower level, such as less than 10,000 copies/ml, less than 7500 or 5000 copies/ml, including less than 2500, 1000, 750, 500, 250, 100 or 50 copies/ml, during a period of withdrawal from retroviral treatment.

The invention method is also advantageous in delaying the rebound to an unacceptably high viral load, such as a viral load of >10,000, >15,000, >20,000, >50,000, >75,000 or >100,000 copies/ml, during the initial period or subsequent periods of withdrawal from retroviral treatment. Rebound to an unacceptably high viral load can be delayed, for example, by at least about 2 weeks, at least about 4, 6, 8, or more weeks, including several months, years or indefinitely, by practice of the invention method.

Yet another contemplated advantage of the invention method is a more rapid or more sustained increase in HIV-specific CD4 T cell counts, as compared to methods that involve withdrawal from anti-retroviral treatment alone.

A further contemplated advantage of the invention method is a reduction or delay in the development of one or more symptoms of acute HIV infection. The symptoms of acute HIV infection are well known in the art and include, for example, fever, headaches, sore throat, pharyngitis, generalized lymphadenopathy and rashes.

Additionally, contemplated advantages of the invention method include a reduction or delay in the development of AIDS symptoms, including AIDS-related opportunistic infections, and an extension of patient survival.

Further contemplated advantages are a higher degree of patient compliance with treatment, a lower cost of treatment, and a lower percentage of patients developing drug resistant strains of virus.

Additionally, it is expected that treatment by the invention method will result in fewer toxic side effects associated with long-term anti-retroviral drug treatment, including a reduction in cardiotoxicity, hyperlipidemia, hyperglycemia, lipodystrophy, insulin resistance, and other adverse effects described in the art.

The following examples are intended to illustrate but not limit the present invention.

### EXAMPLE I

This example shows that therapeutic immunization with an HIV immunogen provides for an unexpectedly large decrease in viral load during a second period of anti-retroviral treatment withdrawal.

Eight chronically infected patients who were virologically suppressed on HAART (highly active anti-retroviral therapy) regimens, who previously had received REMUNE™ therapeutic immunizations were enrolled in an open label prosective study of structured treatment interruption (STI) of HAART.

Lymphocyte proliferative responses (LPR) to HIV p24 antigen, which is a measurement of CD4+ T helper cell activity, were assayed on fresh peripheral blood mononuclear cells (PBMCs). The baseline anti-p24 lymphocyte stimulation index (LSI), and LSI after the indicated number of REMUNE™ immunizations, are shown in Table 1.

TABLE 1

| | Number of | Pre- | Post- |
|---|---|---|---|
| | REMUNE ™ | immunization | immunization |
| Patient | Immunizations | p24 LSI | p24 LSI |
| 1 | 6 | 9.6 | 31.48 |
| 2 | 9 | 2.55 | 26.6 |
| 3 | 6 | 1.63 | 94.09 |
| 4 | 6 | 0.450 | 43.35 |
| 5 | 5 | 2.58 | 22.46 |
| 6 | 10 | 1.55 | 59.67 |
| 7 | 10 | 6.47 | 44.78 |
| 8 | 9 | 1.09 | 12.61 |

The immunized patients were placed on a protocol in which HAART was withdrawn for a maximum of 8 weeks, after which patients were placed back on HAART for another 8 weeks. If patients during the first or second treatment interruption had viral loads >20,000 for three consecutive time points, patients were required to be placed back on HAART.

During the first STI, ⅜ REMUNE™ treated patients displayed viral load (VL) peaks of <10,000 copies/ml. Of note, ⅝ patients decreased their viral load from the peak viral load during the first STI. This median post peak low is consistent with immune control being initiated during the first STI. The patients were then placed back on HAART.

With immune activation of CD4 cells by immunization, and CD8 cells by autologous virus in combination with CD4 help, further immune control was then realized during the second STI, with a lower peak viral load. More specifically, during the second STI, with a mean follow up of 7.5 weeks off HAART, ⅝ of the REMUNE™ patients obtained virological peaks of <10,000 copies/ml. ⅝ patients decreased their viral load from the peak viral load during the second STI.

The peak and post-peak viral loads during the first and second anti-retroviral treatment withdrawal periods (STIs) for the 8 patients are shown in Table 2.

TABLE 2

| | | Post-Peak | | Post Peak |
|---|---|---|---|---|
| | Peak VL | Low VL | Peak VL | Low VL |
| | (copies/ml) | (copies/ml) | (copies/ml) | (copies/ml) |
| Patient | 1^{st} STI | 1^{st} STI | 2^{nd} STI | 2^{nd} STI |
| 1 | 50 | 50 | 67 | 67 |
| 2 | 7205 | 2204 | 1882 | 681 |
| 3 | 165580 | 25177 | 10672 | 7272 |
| 4 | 180606 | 6913 | 9138 | 9138 |
| 5 | 13750 | 2534 | 646 | 228 |
| 6 | 7699 | 2104 | 6267 | 1100 |
| 7 | 62659 | 62659 | 16044 | 2956 |
| 8 | 87233 | >75000 | >75000 | >75000 |
| Mean | 65600 | 22080 | 14960 | 12060 |

CD4 helper p24 LPR responses induced by immunization were observed to be stable during the study, with little variation in mean LSI observed during the first STI, second STI, and intervening treatment period.

In order to determine whether immunization was involved in the lower peak viral load setpoint during the second STI, a least squares regression model was used to examine the relationship between the post immunization baseline LPR responses and the difference between the first and second viral load peaks. A trend was observed suggesting that baseline post-immunization p24 LPRs predicted the decrease in viral load peaks between the first and second STIs (Least Squares slope=−1753.6 p=0.10). Of note, the patient with the lowest T helper baseline LPR response to p24 antigen induced by immunization had the least control of viral replication (Patient 8).

These results suggest that immunological control resulting from HIV therapeutic immunization is involved in the decreased viral load peak observed after the second STI in HIV-infected patients. More specifically, these results suggest that an immunization protocol that enhances HIV specific T helper cell activity (LPR) provides support for CD8 T killer cells, which are activated by autologous virus during the first STI. By combining therapeutic vaccination which stimulates CD4 T helper activity with an initial anti-retroviral treatment interruption period to activate CD8 T cells, viral replication can be maintained below the level that causes clinical disease during subsequent interruption periods. Therefore, such a method is expected to be beneficial in limiting the toxicities, costs, compliance problems and development of drug resistance associated with chronic antiviral drug therapy.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

What is claimed is:

1. A method of treating an HIV-infected individual, comprising:
(a) treating an HIV-infected individual with at least one anti-retroviral compound;
(b) immunizing said individual with an HIV immunogenic composition;
(c) withdrawing treatment with said anti-retroviral compound;
(d) reinitiating treatment with at least one anti-retroviral compound;
(e) repeating step (c) at least once; and
(f) optionally repeating step (d) at least once.

2. The method of claim 1, wherein said immunization induces an anti-HIV CD4+ T helper cell response.

3. The method of claim 1, wherein said immunization comprises administering said HIV immunogenic composition more than once.

4. The method of claim 1, wherein said HIV immunogenic composition comprises a whole-killed HIV virus devoid of outer envelope protein gp120.

5. The method of claim 1, wherein said HIV immunogenic composition comprises an adjuvant.

6. The method of claim 5, wherein said adjuvant comprises incomplete Freund's adjuvant

7. The method of claim 1, wherein said HIV immunogenic composition comprises at least one immunostimulatory sequence (ISS).

8. The method of claim 1, wherein said HIV immunogenic composition is REMUNE™.

9. The method of claim 1, wherein said HIV immunogenic composition is a combination of REMUNE™ and at least one ISS.

10. The method of claim 1, wherein said anti-retroviral compound is selected from the group consisting of a protease inhibitor, a reverse transcriptase inhibitor and a ribonucleotide reductase inhibitor.

11. The method of claim 1, wherein said anti-retroviral compound is selected from the group consisting of a viral adsorption inhibitor, an HIV entry inhibitor, an integrase inhibitor and a virus-cell fusion inhibitor.

12. The method of claim 1, wherein said anti-retroviral treatment in step (a) reduces HIV viral load to less than 5000 copies/ml.

13. The method of claim 1, wherein said anti-retroviral treatment in step (a) reduces HIV viral load to less than 500 copies/ml.

14. The method of claim 1, wherein said anti-retroviral treatment in step (a) reduces HIV viral load to less than 50 copies/ml.

15. The method of claim 1, wherein said withdrawal in step (c) is for a period of time until viral load rises to greater than about 100,000 copies/ml.

16. The method of claim 1, wherein said withdrawal in step (c) is for a period of time until viral load rises to greater than about 50,000 copies/ml.

17. The method of claim 1, wherein said withdrawal in step (c) is for a period of time until viral load rises to greater than about 20,000 copies/ml.

18. The method of claim 1, wherein said withdrawal in step (c) is for a period of at least 2 weeks.

19. The method of claim 1, wherein said withdrawal in step (c) is for a period of about 8 weeks.

20. The method of claim 1, wherein reinitiating said anti-retroviral treatment in step (d) reduces HIV viral load to less than 5000 copies/ml.

21. The method of claim 1, wherein reinitiating said anti-retroviral treatment in step (d) reduces HIV viral load to less than 500 copies/ml.

22. The method of claim 1, wherein reinitiating said anti-retroviral treatment in step (d) reduces HIV viral load to less than 50 copies/ml.

23. The method of claim 1, wherein said reinitiated anti-retroviral treatment in step (d) is for a period of about 8 weeks.

24. The method of claim 1, wherein HIV viral load in said individual following step (e) is maintained at less than about 10,000 copies/ml for a period of at least about 8 weeks.

25. The method of claim 1, wherein HIV viral load in said individual following step (e) is maintained at less than about 5,000 copies/ml for a period of at least about 8 weeks.

26. The method of claim 1, wherein HIV viral load in said individual following step (e) is maintained at less than about 500 copies/ml for a period of at least about 8 weeks.
